(19) United States
(12) Patent Application Publication
(10) Pub. No.: US 2004/0105890 A1
(43) Pub. Date: Jun. 3, 2004

(54) Biocompatible injectable materials

(75) Inventors: Dean A. Klein, North Oaks, MN (US); James D. Brazil, Braham, MN (US)

Correspondence Address: FAEGRE & BENSON LLP, 2200 WELLS FARGO CENTER, 90 SOUTH 7TH STREET, MINNEAPOLIS, MN, 55402, US

(73) Assignee: Carbon Medical Technologies, Inc., St. Paul, MN

(21) Appl. No.: 10/719,530

(22) Filed: Nov. 21, 2003

Related U.S. Application Data
(63) Continuation-in-part of application No. 10/446,647, filed on May 28, 2003.
(63) Continuation-in-part of application No. 10/280,163, filed on Oct. 25, 2002.
(63) Continuation-in-part of application No. 10/212,837, filed on Aug. 6, 2002.
(60) Provisional application No. 60/383,766, filed on May 28, 2002.

(51) Int. Cl. A61K 9/14

(52) U.S. Cl. 424/489

(57) ABSTRACT

The present invention provides a biocompatible injectable material that may be used in a variety of medical applications, including tissue marking, tissue modifying and embolizing procedures.

### CROSS-REFERENCE TO RELATED APPLICATIONS

This application is a continuation-in-part of U.S. application Ser. No. 10/446,647, filed May 28, 2003, incorporated herein by reference, which claims the benefit of U.S. Provisional Application Serial No. 60/383,766, filed May 28, 2002. This application is also a continuation-in-part of U.S. patent application Ser. No. 10/280,163, filed Oct. 25, 2002, incorporated herein by reference, which is a continuation-in-part of U.S. application Ser. No. 10/084,240, filed Feb. 27, 2002. This application is also a continuation-in-part of U.S. patent application Ser. No. 10/212,837, filed Aug. 6, 2002, incorporated herein by reference.

### BACKGROUND

It has been reported that biocompatible microparticles having exposed carbon surfaces may be incorporated into injectable materials for delivery to an anatomical site. For example, U.S. Pat. Nos. 6,394,965, 6,277,392, 5,451,406 and 6,355,275, report the use of such microparticles for tissue marking, tissue modifying and embolizing techniques. Advantageously, the exposed carbon surface provides a biocompatible and substantially non-degradable particle for delivery to an anatomical site.

Although injectable materials utilizing the particles reported in these patents have many advantageous characteristics, it would be further beneficial to provide an injectable material of this nature with enhanced delivery characteristics.

### SUMMARY OF THE INVENTION

In one embodiment, the present invention provides an injectable material including biocompatible microparticles having a major dimension of less than about 100 microns and including an exposed surface of carbon. In particular embodiments, a substantial portion of the microparticles have a major dimension between about 1 and less than about 100 microns, more particularly between about 50 and less than about 100 microns, even more particularly between about 80 and less than about 100 microns. In an alternate embodiment, a substantial portion of the microparticles may have a major dimension between about 10 and about 90 microns, more particularly, between about 50 and about 90 microns, and even more particularly, between about 75 and about 90 microns. In certain embodiments, the injectable material may further include microparticles having a major dimension of greater than about 100 microns.

In another embodiment, the present invention provides a method of marking an anatomical site, in which an injectable material including biocompatible microparticles having a major dimension of less than about 100 microns, and including an exposed surface of carbon, is delivered to the anatomical site. The injectable material may be delivered to, for example, a breast biopsy, colon biopsy, lesion removal or epidermal site.

In a further embodiment, the present invention provides a method of modifying an anatomical site in which an injectable material including biocompatible microparticles having a major dimension of less than about 100 microns, and having an exposed surface of carbon, is implanted in the vicinity of the anatomical site.

In yet another embodiment, the present invention provides a method of embolization, in which an injectable non-magnetic material including biocompatible microparticles having a major dimension of less than about 100 microns, and an exposed surface of carbon, is injected into a blood vessel.

### DETAILED DESCRIPTION

Embodiments of the present invention generally provide an injectable material, which includes biocompatible microparticles having an exposed surface of carbon. A substantial portion of the microparticles have a major dimension of less than about 100 microns.

In one embodiment, the microparticles may include a substrate coated with carbon. Examples of suitable substrate materials include both magnetic materials and non-magnetic materials, including iron, ceramic materials such as zirconium, aluminum oxide or silicon dioxide, gold, titanium, silver, stainless steel, graphite, metal oxides and polymeric materials, as well as alloys, derivatives and combinations thereof. In other embodiments, the microparticles may include solid particulate carbon materials. Suitable carbon materials for these embodiments include, for example, pyrolytic carbon, vitreous carbon, diamond-like carbon, graphite or carbon resins. Combinations of particulate carbon and carbon coated substrates may also be suitable for use in certain embodiments.

The atomic structure of pyrolytic and vitreous carbon is similar to graphite, but the alignment between hexagonal planes of atoms is not as well ordered as in graphite. Pyrolytic carbon is characterized by a more chaotic atomic structure and better bonding between layer planes. The carbon coating may provide a relatively smooth surface for injection into a anatomical site.

Pyrolytic carbon may be produced and coated onto particulate substrate surfaces by known methods. In one technique, hydrocarbons and alloying gases are decomposed to prepare a pyrolytic carbon coating on the particulate substrates. The particulate substrates are contacted with the hydrocarbons and alloying gases in a fluidized or floating bed at a temperature sufficient to cause deposition of pyrolyzed carbon onto the particulate substrate surfaces, typically from about 900 to 1500° C. Inert gas flow is used to float the bed of particulate substrates, optionally including an inert mixing media. The hydrocarbon pyrolysis results in a high carbon, low hydrogen content carbon material being deposited as a solid layer of material onto the particulate substrates.

Alternatively, a carbon coating (sometimes referred to as “ultra-low-temperature isotropic carbon”) may be applied to particulate substrates using any one of other various coating processes for depositing carbon, such as a vacuum vapor deposition process. Such a method uses ion beams generated from any of a variety of known processes, such as the disassociation of CO₂, reactive dissociation in vacuum of a hydrocarbon as a result of a glow discharge, sublimation of a solid graphite source, or cathode sputtering of a graphite source. Gold has been found to be an especially suitable particulate substrate for vacuum vapor deposited carbon. Other particulate substrates, including but not limited to nickel, silver, stainless steel, zirconium, graphite or titanium are also quite acceptable for this type of coating process.

Isotropic carbon may also be applied to temperature-sensitive substrates using physical vapor deposition techniques. Physical vapor deposition involves transferring groups of carbon atoms from a pyrolytic carbon target to a desired substrate at low temperatures. The process may be carried out in high-vacuum conditions to prevent chemical reaction. This technique may be suitable for coating a variety of substrates such as temperature-sensitive polymers and metal alloys.

The high strength, resistance to breakdown or corrosion, and durability of a carbon surface ensures effective, long term functioning of carbon particles in anatomical delivery applications. The established biocompatibility of carbon such as pyrolytic and vitreous carbon makes the described particles particularly suitable as injectable materials. In one embodiment, the particulate substrates may be completely encased by a carbon surface. This results in a uniformly coated particle with no substrate exposure on the surface of the particle. Preferred carbon coatings may be in the range of fractions of thousandths of an inch, e.g., about 5 ten-thousands of an inch (0.0005 inches), on average, covering the surface of the particle substrate. In another embodiment, microparticles of pyrolytic carbon (without a substrate) may be formed by removing carbon deposits from substrates and then grinding the deposits to a desired size. The particles may also be subjected to a cleaning, polishing and sieving process to remove contaminants, smooth the particle surface to a desired texture and to separate out particles of a size less than or greater than a desired size range. The size range of the microparticles may be narrowly tailored as desired for a specific application by utilizing standard sieving procedures.

The particles may be shaped and sized to provide enhanced passage through a hypodermic needle. The shape and size of the injected particles may be varied to enhance the flow of the particles during injection. A substantial portion of the microparticles incorporated into embodiments of the present invention may have a major dimension of less than about 100 microns, more particularly from the sub-micron level to less than about 100 microns, even more particularly between about 1 and less than about 100 microns, even more particularly between about 50 and less than about 100 microns and even more particularly between about 80 and less than about 100 microns. In an alternate embodiment, the microparticles may have a major dimension between about 10 and about 90 microns, more particularly, between about 50 and about 90 microns, and even more particularly, between about 75 and about 90 microns. These microparticles may also be combined with particles having a major dimension of greater than 100 microns in certain embodiments. In one embodiment, the concentration of particles having a major dimension of less than 100 microns may be greater than about 50 w/w percent, more particularly, greater than about 75 w/w %.

Optionally, the biocompatible microparticles may be delivered to the anatomical site in a suitable biocompatible carrier fluid. Any biocompatible carrier fluid that can deliver the microparticles to an anatomical site may be used in accordance with the present invention. A carrier fluid may be a biologically compatible solution. Examples of suitable carrier fluids include solutions containing glucan, collagen, saline, dextrans, glycerol, polyethylene glycol, corn oil or safflower, other polysaccharides or biocompatible polymers, methyl cellulose, agarose, hemostatic agents or combinations thereof. In certain embodiments, a curable polymer such as PMMA may be added to the carrier to provide additional stiffening characteristics. The viscosity of the carrier may range between about 10 and 75,000 centipoise.

Solutions containing β-glucan and collagen are particularly suitable carrier fluids for the present invention. β-glucan is a naturally occurring constituent of cell walls in essentially all living systems including plants, yeast, bacteria, and mammalian systems. Its effects and modulating actions on living systems have been reported by Abel et. al., “Stimulation of Human Monocyte B-glucan Receptors by Glucan Particles Induces Production of TNF-∂ and 1L-B,” Int. J. Immunopharmacol., 14(8):1363-1373, 1992. β-glucan, when administered in experimental studies, elicits and augments host defense mechanisms including the steps required to promote healing, thereby stimulating the reparative processes in the host system. β-glucan is removed from tissue sites through macrophagic phagocytosis or by enzymatic destruction by serous enzymes. The degradation or removal of β-glucan, as well as its available viscosity and lubricous nature, make it a useful carrier for the particles in anatomical delivery applications.

Aqueous solutions of β-glucan may be produced that have favorable physical characteristics as a carrier liquid for embodiments of the present invention. The viscosity can vary from a thin liquid to a firm, self-supporting gel. Irrespective of viscosity, the β-glucan solution has excellent lubricity, thereby creating an injectable material which is easily administered by delivery to a predetermined anatomical site through a small bore needle. Useful β-glucan compositions include β-D-glucans containing 4-0-linked-β-D-glycopyranosyl units and 3-0-linked-β-D-glycopyranosyl units, or 5-0-linked-β-D-glycopyranosyl units and 3-0-linked-β-D-glycopyranosyl units. The carrier may be of sufficient viscosity to assure that the particles remain suspended therein, for a sufficient time duration to accomplish the injection procedure.

Collagen, another suitable carrier, is a naturally occurring protein that provides support to various parts of the human body, including the skin, joints, bone and ligaments. One suitable injectable collagen manufactured by the McGhan Medical Corporation, Santa Barbara, Calif., and sold under the trade names ZYDERM and ZYPLAST, is derived from purified bovine collagen. The purification process results in a product similar to human collagen. Collagen solutions may be produced within a wide viscosity range to meet an individual patient's needs, and mixed with the particulate material for injection into a patient.

Another example of a suitable carrier fluid is a solution containing methyl cellulose or another linear unbranched polysaccharide. Further examples of appropriate carrier fluids include agarose, hyaluronic acid, polyvinyl pyrrolidone or a hydrogel derivative thereof, dextran or a hydrogel derivative thereof, glycerol, polyethylene glycol, oil-based emulsions such as corn or safflower, or other polysaccharides or biocompatible organic polymers either singly or in combination with one or more of the above-referenced solutions.

The injectable material may also include a biologically active agent, such as biologically active liquid or gel. For example, the biologically active agent may include an anti-inflammatory agent, anti-microbial agent, a hemostatic agent, a biocompatible adhesive agent, or a cell-derived agent.

The amount of particles in the injectable material may be any amount that will provide a material that is flowable and injectable, and that will allow a desired amount of particles to be delivered to an anatomical site. Amounts of particles in the material can be in the range from about 5 to 85 percent by volume, more particularly from about 20 to 60 percent by volume, and most particularly from about 20 to 50 percent by volume.

In use, the injectable material will typically be injected as a slurry, suspension, or emulsion in a carrier through a needle, into an anatomical site. The injectable material may be delivered to a site using any instrument or apparatus that can be used to inject an amount of microparticles, preferably contained or suspended in a carrier, through the skin or mucosa, to a desired site. Suitable instruments include hypodermic needles or other similar needle-like apparatuses, such as any small bore instrument, cannula, etc. (All of these types of instruments will be referred to collectively herein, for convenience, using the term “hypodermic needle” or “needle.”) The particular instrument used for delivery is not critical, provided that its components are compatible with the injectable material.

Advantageously, the injectable materials formed according to embodiments of the present invention provide for enhanced delivery to anatomical sites. More particularly, injectable materials formed according to embodiments of the present require substantially less force to expel the materials through a needle or similar surgical instrument. This may allow a clinician to more accurately, easily and effectively deliver the injectable material to an anatomical site. Furthermore, embodiments of the present invention may be more easily expelled through smaller-diameter needles than materials having substantial amounts of particles of 100 microns and greater, more particularly, 90 microns or greater. The ability to utilize smaller needles may provide clinicians with more precision in delivering the injectable material, and may result in an even less invasive medical procedure.

According to one example, the injectable material may be delivered using a hypodermic needle and a syringe, by inserting the hypodermic needle at, or in the vicinity of, a desired site, followed by delivery of the injectable material to the site. Once a needle is placed, the injectable material may be slowly injected through the needle to the desired site. As previously noted, the particles are of a size that may provide for improved delivery through a hypodermic needle or like instrument.

The amount of microparticles introduced to the anatomical site may be any amount sufficient to achieve the desired result. The amount delivered may vary depending on factors such as the specific procedure, the size and shape of the microparticles, and other factors particular to specific patients. Such factors will be within the skill of an artisan of ordinary skill in the medical arts, and such an artisan will be able to understand what is a useful amount of particles for delivery to anatomical sites.

The injectable material of the present invention may be suitable for use in a variety of applications. Suitable applications include embolization of blood vessels, tissue marking for the identification of anatomical sites and tissue modifying of anatomical sites, particularly urinary and anal sphincters.

For example, U.S. Pat. No. 6,394,965 to Klein, incorporated herein by reference, reports tissue marking methods that incorporate microparticles having a size range of about 100 microns and larger. U.S. Pat. No. 6,277,392 to Klein and U.S. Pat. No. 5,451,406 to Lawin et al., each incorporated herein by reference, report methods of modifying the urinary and anal sphincters of patients by delivering microparticles having a size range of about 100 microns and larger. U.S. Pat. No. 6,355,275, incorporated herein by reference, reports methods for embolizing blood vessels by delivering microparticles having a size range of about 100 microns and larger. U.S. application Ser. No. 10/446,647, filed May 28, 2003 and incorporated herein by reference, reports magnetic particles having a size range of about 80 microns and larger. U.S. patent application Ser. No. 10/280,163, filed Oct. 25, 2002 and incorporated herein by reference, reports methods of modifying the lower esophageal sphincter by delivering microparticles having a size ranging from the sub-micron level to substantially greater than about 100 microns. U.S. patent application Ser. No. 10/212,837, filed Aug. 6, 2002 and incorporated herein by reference, reports methods of modifying the swallowing system by delivering microparticles having a size ranging from the sub-micron level to substantially greater than about 100 microns. The methods reported in these references may also be performed using embodiments of the injectable materials of the present invention.

In one embodiment for example, the injectable material of the present invention may be suitable for marking anatomical sites. For example, microparticles having a major dimension of less than about 100 microns may be suitable for marking an anatomical site, and may then be carried away after a period of time. In another example, the injectable material may be delivered to an anatomical site for substantially permanent marking. Whether the particles remain permanently at the anatomical site depends on the size of the particles, as well as the physiology of the anatomical site.

In another embodiment, the injectable material of the present invention may be used to modify anatomical sites, such as an anatomical sphincter or a patient's swallowing system. For example, microparticles having a major dimension of less than 100 microns may be suitable to modify tissue substantially permanently, while still providing improved delivery characteristics when compared to modifiers including substantial portions of microparticles with a major dimension above about 100 microns.

In yet another embodiment, the injectable material of the present invention may be used as an embolizing material in a blood vessel. For example, microparticles having a major dimension of less than about 100 microns may be suitable for substantially permanent embolization, while still providing improved injectability characteristics when compared to modifiers having microparticle sizes above about 100 microns. The size of the particles delivered to the blood vessel will vary depending upon the diameter of the blood vessel.

As is evident from the foregoing, the injectable material of the present invention may be formed with microparticles of various size ranges depending on the intended medical application. These size ranges may be uniquely tailored to achieve optimal results for a given application, while still providing for improved delivery characteristics. For example, as reported in the Examples below, injectable materials having substantial amounts of microparticles of less than about 90 microns require less needle expulsion force than microparticles having a major dimension of about 90 microns and greater.

### EXAMPLE 1

Injectable materials A, B and C were loaded into a series of 20 XXTW (0.030 inch inner diameter) needles of varying lengths obtained from HART Enterprises, Sparta, Mich. Material A contained carbon coated particles having a major dimension between about 63 and 75 microns. Material B contained carbon coated particles having a major dimension between about 75 and 90 microns. Material C contained carbon coated particles having a major dimension between about 90 and 105 microns. The size range of materials A-C were obtained by sieving the particles employing standard sieving procedures. Materials A, B and C also included a sufficient amount of a 3-glucan carrier such that the particle-to-carrier ratio was substantially equal for each material. Each material was then expelled out of a needle into air by applying pressure to the needle plunger using a calibrated compression gauge to determine the force (in grams) required for particle expulsion. Table 1 shows the results of the experiment.

TABLE 1

| Needle Length (in) | Material A (g) | Material B (g) | Material C (g) |
|---|---|---|---|
| 1.5 | 594 | 574 | 699 |
| 5 | 1385 | 1378 | 1578 |
| 10 | 2446 | 2529 | 2782 |

Table 1 demonstrates that materials A and B were more easily expelled than material C.

### EXAMPLE 2

Materials A, B, and C as reported in Example 1 were each loaded into a series of 21 TW (0.023 in. inner diameter) needles (Hart Enterprises) and expelled into air as in Example 1. The results are shown in Table 2.

TABLE 2

| Needle Length (in) | Material A (g) | Material B (g) | Material C (g) |
|---|---|---|---|
| 1.5 | 794 | 722 | 866 |
| 5 | 1936 | 1849 | 2167 |
| 10 | 3251 | 3278 | 3749 |

Table 2 demonstrates that materials A and B were more easily expelled than material C.

### EXAMPLE 3

Materials A, B, and C as reported in Example 1 were each loaded into a series of 22 TW (0.020 in. inner diameter) needles (Hart Enterprises) and expelled into air. The results are shown in Table 3.

TABLE 3

| Needle Length (in) | Material A (g) | Material B (g) | Material C (g) |
|---|---|---|---|
| 1.5 | 952 | 883 | 1065 |
| 5 | 2327 | 2270 | 2689 |
| 10 | 4591 | 4194 | 4779 |

Table 3 demonstrates that materials A and B were more easily expelled than material C.

### EXAMPLE 4

Materials A, B, and C as reported in Example 1 were each loaded into a series of 23 TW (0.017 in. inner diameter) needles (Hart Enterprises) and expelled into air. The results are shown in Table 4.

TABLE 4

| Needle Length (in) | Material A (g) | Material B (g) | Material C (g) |
|---|---|---|---|
| 1.5 | 1151 | 1181 | 1336 |
| 5 | 2914 | 2760 | 3327 |
| 10 | 5318 | 5088 | 5935 |

Table 4 demonstrates that materials A and B were more easily expelled than material C.

### EXAMPLE 5

Materials A, B, and C as reported in Example 1 were each loaded into a series of 25 gauge (0.011 in. inner diameter) needles (Hart Enterprises) and expelled into air. The results are shown in Table 5.

TABLE 5

| Needle Length (in) | Material A (g) | Material B (g) | Material C (g) |
|---|---|---|---|
| 3.5 | 3588 | plugged | not attempted |

Table 5 demonstrates that only material A was able to be expelled through the 25 gauge needle.

The examples demonstrate that injectable materials that do not include microparticles having a major dimension of 100 microns or greater are more easily expelled from needles than materials having microparticles of 100 microns or greater. Injectable materials with microparticles of 90 microns or less may have particularly advantageous delivery characteristics.

1. A method of modifying an anatomical site comprising:
injecting into the anatomical site a tissue modifying material comprising biocompatible microparticles having a major dimension of less than about 100 microns and including an exposed surface of carbon.

2. The method of claim 1 wherein the microparticles have a major dimension between about 1 and less than about 100 microns.

3. The method of claim 1 wherein the microparticles have a major dimension between about 50 and less than about 100 microns.

4. The method of claim 1 wherein the microparticles have a major dimension between about 80 and less than about 100 microns.

5. The method of claim 1 wherein the microparticles have a major dimension between about 10 and about 90 microns.

6. The method of claim 1 wherein the microparticles have a major dimension between about 50 and about 90 microns.

7. The method of claim 1 wherein the microparticles have a major dimension between about 75 and about 90 microns.

8. The method of claim 1 wherein the injectable material further comprises a carrier fluid.

9. The method of claim 1 wherein the injectable material further comprises a biologically active agent.

10. The method of claim 1 wherein the anatomical site comprises a swallowing system of a patient.

11. The method of claim 1 wherein the anatomical site comprises a lower esophageal sphincter of a patient.

12. The method of claim 1 wherein the anatomical site comprises a urinary or anal sphincter of a patient.

13. A method of embolization comprising:
injecting into a blood vessel an injectable material comprising biocompatible microparticles having a major dimension of less than about 100 microns and including an exposed surface of carbon.

14. The method of claim 13 wherein the biocompatible microparticles have a major dimension between about 80 and less than about 100 microns.

15. A method of marking an anatomical site comprising:
injecting into the anatomical site an injectable material comprising biocompatible microparticles having a major dimension of less than about 100 microns and including an exposed surface of carbon.

16. The method of claim 15 wherein the injectable material is delivered to a breast biopsy, colon biopsy, lesion removal or epidermal site.

17. The method of claim 15 wherein the microparticles have a major dimension between about 1 and less than about 100 microns.

18. The method of claim 15 wherein the microparticles have a major dimension between about 50 and less than about 100 microns.

19. The method of claim 15 wherein the microparticles have a major dimension between about 80 and less than about 100 microns.

20. The method of claim 15 wherein the microparticles have a major dimension between about 10 and about 90 microns.

21. The method of claim 15 wherein the microparticles have a major dimension between about 50 and about 90 microns.

22. The method of claim 15 wherein the microparticles have a major dimension between about 75 and about 90 microns.

23. The method of claim 15 wherein the injectable material further comprises a carrier fluid.

24. An injectable anatomical marking material comprising biocompatible microparticles having a major dimension of between about 50 and about 90 microns, and including an exposed surface of carbon.

25. The marking material of claim 24 wherein the particles have a major dimension of between about 75 and about 90 microns.

26. An injectable anatomical modifying material comprising biocompatible microparticles having a major dimension of between about 50 and about 90 microns, and including an exposed surface of carbon.

27. The modifying material of claim 26 wherein the particles have a major dimension of between about 75 and about 90 microns.

28. An injectable embolization material comprising biocompatible microparticles having a major dimension of between about 50 and about 90 microns, and including an exposed surface of carbon.

29. The embolization material of claim 28 wherein the particles have a major dimension of between about 75 and about 90 microns.
